(19) United States
(12) Patent Application Publication
(10) Pub. No.: US 2005/0026813 A1
(43) Pub. Date: Feb. 3, 2005

(54) Antibiotic-metal complexes in the detection of gram-positive bacteria and other biological analytes

(76) Inventors: Alan D. Olstein, Mendota Heights, MN (US); Joellen Feirtag, St. Paul, MN (US)

Correspondence Address: REED INTELLECTUAL PROPERTY LAW GROUP, 800 MENLO AVENUE, SUITE 210, MENLO PARK, CA, 94025, US

(21) Appl. No.: 10/631,341

(22) Filed: Jul. 30, 2003

(51) Int. Cl. A61K 38/16; A61K 31/555; C07K 9/00; C07F 5/00

(52) U.S. Cl. 514/6; 514/35; 514/184; 530/322; 534/15

(57) ABSTRACT

Complexes of antibiotics and metals are provided that are useful in detecting microorganisms, including gram-positive bacteria, Mycobacteria, permeabilized gram-negative bacteria, protozoans and other biological analytes, and are particularly useful in detecting gram-positive bacteria. The complexes are preferably chelated complexes wherein the antibiotic is a glycopeptide antibiotic, quinolone antibiotic, ribonucleoside antibiotic, mixtures thereof, and analogs and derivatives thereof, and (b) a detectable label comprising a transition or lanthanide metal. The complexes provide for chemiluminescent, fluorescent or magnetic detection of the analyte. Methods of synthesizing and using the complexes are also provided.

### FIELD OF THE INVENTION

The invention relates generally to detection of biological analytes, and more particularly relates to novel complexes of antibiotics and metals useful in the catalytic detection of gram-positive bacteria, permeabilized gram-negative bacteria, Mycobacteria, protozoans and other biological analytes.

### BACKGROUND

The risk from pathogenic microorganisms in foods has been recognized for many years, and bacterial agents are generally implicated as the contaminants. Food-borne disease may be one of the most notable public health problems. The rapid detection and identification of pathogenic microorganisms in foods, and its manufacturing environment, is of utmost importance in the development and implementation of control and prevention strategies leading to a safer food supply. Bacterial pathogens account for the greatest percentage of reported outbreaks of food-borne illnesses. For example, a predominant cause in reported cases is Salmonella Enteritidis, thought to originate in egg products. Additionally, multi-state outbreaks of Escherichia coli contribute significantly to the total figures for morbidity and mortality. Listeria monocytogenes, a gram-positive contaminant, is an emerging public health threat to the safety of food products as well.

Antibiotic-resistant bacteria, including gram-positive bacteria, are becoming an increasing issue in hospitals and communities. Community-acquired pneumonia, the result of infection with Streptococcus pneumoniae, strikes an increasing number of individuals. Unfortunately, resistance to penicillin, the most common agent used to treat S. pneumoniae, is also on the incline. Additional resistance has been reported against cephalosporins and non-beta-lactam agents, and nearly half of these strains can be classified as highly resistant. High-dose penicillin and cephalosporins remain first-line therapies, however, a broader range of agents is needed. The development of vancomycin, the next generation of fluoroquinolones, and agents such as sparfloxacin, the new streptogramin class, as well as combination therapies, is providing one means of treating resistant pneumococci.

The gram-positive pathogens, penicillin-resistant S. pneumoniae, methicillin-resistant Staphylococcus aureus and vancomycin-resistant enterococci, complicate the treatment of serious infections and have been linked to extended hospitalizations, higher medical costs and high mortality rates. Drug-resistant S. pneumoniae poses a growing threat to people in places where they live and work since S. pneumoniae infections—including pneumonia, sinusitis, meningitis and otitis media—are among the leading causes of death and illness among the elderly, young children and persons with underlying medical conditions.

S. aureus, the most common cause of more than a dozen conditions in both hospitals and communities, often colonizes without any sign of infection, and then from this reservoir gains access to skin and deep tissue, where it subverts the immune system. Staphylococcal infections range from local skin infections to endocarditis (heart valve infection), osteomyelitis (bone infection) and sepsis (blood stream infection). Methicillin-resistant S. aureus first emerged in the early 1960s and several strains of S. aureus are now resistant to a wide variety of currently available antibiotics, including penicillins, macrolides, fluoroquinolones and lincosamides.

In the same bacteria family, multidrug-resistant Staphylococcus epidermidis also compromises patient health, and has been established as a leading cause of hospital-acquired bloodstream infections. A high percentage of S. epidermidis isolates in hospitals are methicillin resistant, and recent studies have found resistance to quinolones, cephalosporins and vancomycin. This drug resistance is a growing concern, particularly for immunocompromised cancer patients.

Vancomycin is considered the agent of last resort for gram-positive infections. Vancomycin-resistant enterococci, an increasingly frequent cause of hospital-acquired infections, are resistant to virtually all currently available antibiotics including vancomycin.

Accordingly, there is a significant need in the art for an effective method of detecting and diagnosing these pathogens. Unfortunately, current methods of testing bacteria, yeast and fungi are excessively time consuming and labor intensive. While the onset of symptoms may be exceedingly rapid, laboratory based diagnosis can typically take several days. Common techniques used to detect the presence of bacteria involve aseptic transfer of a sample, streaking the sample suspected of having bacterial organisms on agar plates after serial dilution, and colony enumeration. This laborious and lengthy process requires at least 24 to 48 hours for a positive result and substantially longer for a negative result.

The detection and characterization of microbial contaminants in food and water samples also rely upon bacterial enumeration techniques, both in liquid and solid culture media. These methods, while sufficiently sensitive to detect a small number of viable organisms, require lengthy sample preparation time. The use of ELISA techniques and nucleic acid hybridization probes, while accurate, have less sensitivity, and therefore require lengthy isolation and enrichment periods to reach the analytical detection limits for these techniques. Therefore, there is a need for a rapid and sensitive method of determining cell numbers.

Other analyte tests require an organism to ingest a detectable material, such as fluorescein. In yet other tests, an antibody, specific for an antigen on the target bacteria is labeled with fluorescein to make a fluorescent antibody. Chemiluminescent labeling of macromolecules has been demonstrated to yield greater analytical sensitivity than the use of many fluorescent probes because of simplicity of the optics resulting in lower background signal. Another approach involves use of a visualization polymer coupled to a detecting agent that binds the target organism, wherein the visualization polymer is made up of detectable visualization units, such as multiple enzymes or labeled polyolefins, which are directly or indirectly bonded together (see, e.g., U.S. Pat. No. 4,687,732 to Ward et al.). Another approach involves covalent conjugation of polymyxin B (PMB) and an enzyme reporter molecule, such as horseradish peroxidase (HRP), to produce a complex for use in a binding assay to detect the target organism (Applemelk et al., Anal. Biochem. 207:311-316 (1992)). An organic “chemical tag” that comprises populations of binding agents and detectable labels has also been described (U.S. Pat. No. 5,750,357 to Olstein et al.).

While antibiotics have been used primarily as therapeutic agents and growth promoting substances, there is evidence in the literature for their use for diagnostic purposes. See, Appelmelk et al., Anal. Biochem. 207:311-316 (1992), and U.S. Pat. No. 5,750,357 to Olstein et al. Unfortunately, many methods for conjugation of reporter groups to antibiotic compounds are frequently unsuitable, for both technical reasons, such as loss of biological activity, loss of solubility and economic, i.e. the cost of enzymes, dyes and the conjugation chemistry.

Therefore, there remains a need in the art for additional detection methods for microorganisms, especially pathogenic bacteria. Ideal methods would utilize small reporter groups and provide sensitive detection.

### SUMMARY OF THE INVENTION

Accordingly, one aspect of the invention provides a method for the sensitive and rapid detection of bacteria. The present invention is also directed to a novel antibiotic derivatives that takes the form of a chelated complex comprising an antibiotic and a metal. The chelated complexes are useful as bacterial probes having sensitive detection and being capable of detecting low cell numbers. The complexes are also useful to study the development of antibiotic resistance.

The chelated complexes of the present invention are comprised of (a) an antibiotic selected from the group consisting of glycopeptide antibiotics, ribonucleoside antibiotics, and combinations thereof, and (b) a detectable label comprising a transition or lanthanide metal. The complexes bind to gram-positive bacteria or Mycobacteria cells. Permeabilized gram-negative bacteria cells and protozoans may also be tested.

Useful transition metals are Zn, Cu, Ni, Co, Fe, Mn, Cr, Tc, and their isotopes. Lanthanide metals may also be used, such as Eu, Gd, Tb, Dy, Er, Lu, and their isotopes. Preferred metals include Cr and Co, and more preferably Co.

Preferred antibiotics include glycopeptide antibiotics, such as actinoidin, avoparcin, balhimycins, chloroorienticins, daptomycin, ereomycin, galacardin, helevecardin, orienticins, ristocetins, ristomycin A, teicoplanin, vancomycin, and derivatives thereof. Also preferred are ribonucleoside antibiotics, such as the lincosamides, and derivatives thereof. Another preferred class of antibiotics are the quinolones, including the fluoroquinolones as well as derivatives thereof.

Any antibiotic or derivative thereof, that is able to form a complex with a transition or lanthanide metal, and retain the ability to bind to microorganisms, particularly gram-positive bacteria and Mycobacteria cells, and in some embodiments, permeabilized gram-negative bacteria and protozoans, is encompassed within the invention. It is not necessary that the antibiotic be optimized for, or effective at, killing the bacteria or protozoans. Instead, all that is necessary is that the antibiotic (or derivative) in the metal complex retain the ability to bind to the target organism. The metal is then provided at the site of the target microorganism to catalyze the chemiluminescent reaction and provide detection for the target microorganism.

The invention also includes a method for synthesizing a chelated antibiotic-metal complex, comprising: admixing (i) a water soluble salt of a metal selected from the group consisting of transition metals and lanthanides with (ii) an antibiotic selected from the group consisting of glycopeptide antibiotics, ribonucleoside antibiotics, and quinolone antibiotics, in (iii) a solvent for the metal salt and the antibiotic; wherein the admixing is conducted under conditions effective to promote chelation of the metal by the antibiotic, thereby forming a solution of the chelated antibiotic-metal complex. The complex can then optionally be desalted, isolated and dried.

The solvent is preferably an aqueous buffer, for example. Although in certain instances, nonaqueous solutions or mixtures of aqueous and nonaqueous solutions may be used. A preferred method of desalting the complex is by dialysis or gel filtration. Preferred methods of drying the complex include freeze-drying and spray drying.

The invention further includes a method for synthesizing an antibiotic-metal complex, wherein the complex is formed in situ, and need not be isolated for performing assays. The complex may optionally be treated in situ, (e.g., washed) to remove uncomplexed metal and/or antibiotic.

The invention further provides a kit useful for conducting a chemiluminescent assay of microorganisms, comprising: the chelated complex of the invention, a source of peroxide and oxidizable substrate. A preferred antibiotic-metal complex is a vancomycin-cobalt complex.

The invention further provides a method for conducting a chemiluminescent assay of microorganisms in a sample comprising (a) contacting a sample with the chelated complex of the invention, (b) separating complex-bound microorganisms from unbound complex, (c) adding an oxidizable substrate and a source of peroxide to complex-bound microorganisms; and detecting complex-bound microorganisms by measuring luminescence.

Microorganisms that are of particular interest for detection, include gram-positive bacterial cells such as aerobic spore-forming Bacilli, anaerobic spore-forming Bacilli, Listeria, Nocardia, Pneumococci, Staphylococci, and Streptococci. The complexes of the invention may also be used to detect Mycobacteria such as Mycobacterium tuberculosis hominis, M. bovis, M. avium, M. paratuberculosis, and M. leprae. Protozoans, such as Plasmodia and other pathogens, such as permeabilized gram-negative bacteria, can also be detected using other embodiments of the invention.

Additional objects, advantages and novel features of the invention will be set forth in part in the description that follows, and in part will become apparent to those skilled in the art upon examination of the following, or may be learned by practice of the invention.

### BRIEF DESCRIPTION OF THE DRAWINGS

FIG. 1 shows the structure of vancomycin.

FIG. 2 shows the structure of the copper-vancomycin complex.

FIG. 3 shows a predicted structure of the cobalt-vancomycin complex, showing the cobalt interaction with the proximal phenolic hydroxyl groups on residues 5 and 6.

FIG. 4 shows the electronic spectra of the free vancomycin and the complex of vancomycin-Co(II).

FIG. 5 shows a graph of a chemiluminescent cell titration using vancomycin-Co(II) showing luminescence relative to the number of Listeria monocytogenes cells.

### DETAILED DESCRIPTION OF THE INVENTION

1. Definitions and Overview:

Before the present invention is described in detail, it is to be understood that unless otherwise indicated this invention is not limited to specific antibiotics, metals, ligands or the like, as such may vary. It is also to be understood that the terminology used herein is for the purpose of describing particular embodiments only and is not intended to limit the scope of the present invention.

It must be noted that as used herein and in the claims, the singular forms “a,” “and” and “the” include plural referents unless the context clearly dictates otherwise. Thus, for example, reference to “an antibiotic” includes two or more antibiotics, reference to “a complex” includes two or more complexes, and so forth.

“Optional” or “optionally” means that the subsequently described circumstance may or may not occur, so that the description includes instances where the circumstance occurs and instances where it does not. For example, recitation of a chemical modification as “optionally” encompasses both the compound as chemically modified and the unmodified compound.

The term “derivative” refers to compounds having a similar chemical structure, encompassing variations, truncations and substitutions, and includes analogs. For example, the quinolone and fluoroquinolone family are related by a common parent quinolone nucleus having varying R groups. An analog of ciprofloxacin thus would be norfloxacin, wherein the cyclopropyl substituent at the 1 position is replaced with an ethyl substituent. The glycopeptide antibiotics in particular include diverse structures and substituents. For example, a large variation in sugar residues is observed in the compounds showing antibacterial activity. Additional examples of derivatives will be discussed below.

The term “latex” refers to colloidal dispersions of high molecular weight polymer.

The term “microorganism” refers to any microscopic organisms, generally pathogenic organisms, such as bacterial pathogens, protozoans, Mycobacteria, and the like, but not limited to pathogenic species.

The term “pathogen” refers to any microorganism known to induce a disease in an animal, including humans.

The present invention provides for a novel antibiotic-metal chelate constituting a new class of chemiluminescent labels useful for rapid detection of microorganisms, preferably gram-positive pathogens and Mycobacteria, as well as non-pathogenic bacteria, and permeabilized gram-negative bacteria, protozoans and Mycobacteria. The invention further provides for a method of the manufacture of these complexes and a method for their use in a rapid detection assay for pathogens.

Many antibiotics can be rendered into redox-active conjugates with minimal chemical modification, yielding chemiluminescent antibiotic metal chelates useful in the methods of the invention. The antibiotic metal chelates of the invention appear to be as catalytically active as the oxidative enzymes and organo-metallic complexes of the porphyrins, which catalyze the hydrogen peroxide-mediated oxidation of luminol. Most significantly, the chelates are biologically active and are not sterically hindered by large enzymes or conjugated organic groups. In some embodiments, however, it may be desirable to modify the structure of the antibiotic to add a metal chelating ligand.

The present invention relates to the use of these chemiluminescent antibiotic probes for sensitive detection of microorganisms. The invention provides for a detection sensitivity for potential pathogens below 10 cells per sample. Contemporaneous assay of complex samples using immuno-magnetic capture of bacteria coupled with chemiluminescent detection can be performed. By coupling the immuno-magnetic capture technique with sensitive chemiluminescent detection, the analysis time is reduced from days to a few hours.

In addition, these chemiluminescent antibiotic probes can be used to study the uptake and biological affinity of antibiotics by resistant bacteria, compared with sensitive organisms. While not being held to any particular theory, it is hypothesized that there may be a correlation between antibiotic affinity/uptake and the antibiotic resistant state. Several instances of antibiotic resistance have been traced to lesions in the antibiotic uptake systems or metabolic enzymes involved in their metabolism (Vaara et al., FEBS Lett. 129:145-149 (1981), Sutcliffe et al., Antimicrob. Agents and Chemother. 40:1817-1824 (1996); and Gibreel et al., Antimicrob. Agent and Chemother. 42:3059-3064 (1998). By correlating antibiotic binding to microorganisms using standard Minimal Inhibitory Concentration techniques to titration data, a relationship between resistance and antibiotic affinity may be demonstrated. The chemiluminescent antibiotic binding assay can be used to examine the variables in antibiotic resistance acquisition including, time course, environmental influences and effects of microbial flora. However, the antibiotic-metal complexes do not have to provide effective antibiotic activity to be useful in the methods for detecting the microorganisms. Thus, the methods described herein encompass many antibiotic derivatives that may not be effective as therapeutic agents, or that may not be useful because of side effects when used in a living animal. All that is required is that the antibiotic form a stable complex with the metal and retain binding affinity for the microorganism that it is desired to target.

The chelated complexes of the present invention are comprised of (a) an antibiotic selected from the group consisting of glycopeptide antibiotics, ribonucleoside antibiotics, quinolone antibiotics, and combinations thereof, and (b) a detectable label comprising a transition or lanthanide metal. The complex is capable of binding to gram-positive bacterial cells, Mycobacteria cells, permeabilized gram-negative bacterial cells and protozoans.

II. Antibiotics:

The present invention is applicable to any antibiotic capable of binding to gram-positive bacterial cells, Mycobacteria cells, permeabilized gram-negative bacterial cells and protozoans. Preferred target pathogens are gram-positive bacteria. The complexes of the present invention are also capable of binding to permeabilized gram-negative bacterial cells, wherein the outer membrane is permeabilized by treatment with surfactants, cationic compounds such as polylysine and lysozyme, or EDTA, by treating with a pH greater than or equal to pH 11 (e.g., treatment with sublethal levels of trisodium orthophosphate at pH 11, as described in U.S. Pat. No. 6,287,617 to Bender). In addition, pretreatment with the non-toxic fragment of polymyxin B, polymyxin B nonapeptide (PMBN), has been shown to render gram-negative bacteria susceptible to antibiotics that are otherwise unable to pass through the outer membrane envelope. Polymyxin B treatment of itself is non-lethal for the bacteria. The preferred classes of antibiotics are described below.

A. Glycopeptide Antibiotics:

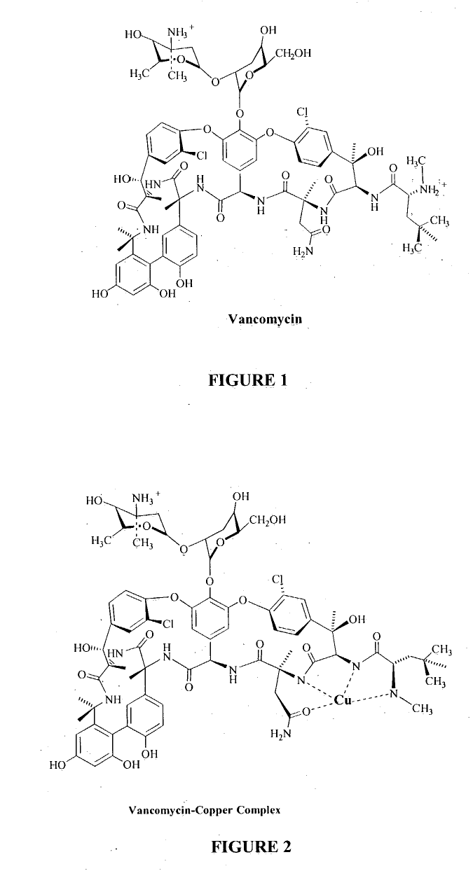

Vancomycin and the related glycopeptide antibiotics are unique antibiotics which bind tightly to crosslinking fragments in the peptido-glycan layer of gram positive bacteria, and inhibit the cross-linking reactions which stabilize the peptido-glycan layer against osmotic pressure fluctuations in the micro-organisms' environment. High levels of this antibiotic are taken up by target organisms, frequently several times the dry weight of the susceptible bacteria. The mechanism of vancomycin action is described in greater detail in Williams et al., Angew. Che. Int. Ed. 38:1172-1193 (1999). The structure of vancomycin is depicted in FIG. 1.

The glycopeptide antibiotics generally consist of a peptide backbone comprised of seven amino acids. Numbered from the N-terminus to the C-terminus, one to seven, residues 4 and 5 are usually a p-hydroxyphenylglycine, residues 2 and 6 are usually tyrosine or tyrosine-like and residue 7 is generally a 3,5 dihydroxyphenylglycine. Residues 1 and 3 are frequently aliphatic amino acids, such as methyl-leucine and asparagine. In other glycopeptide antibiotics, such as ristocetin A and teicoplanin these residues are aromatic amino acids. Further, teicoplanin is a mixture of six analogs: one compound has a terminal hydrogen at one of the bridging oxygen atoms, while five compounds have an R substituent of either a decanoic acid [n-, 8-methyl-, 9-methyl-, (Z)-4-] or of a nonanoic acid [8-methyl], as described in Goodman and Gilman's The Pharmacological Basis of Therapeutics, Tenth Edition, Eds. Hardman and Limbird, page 1264 (2001).

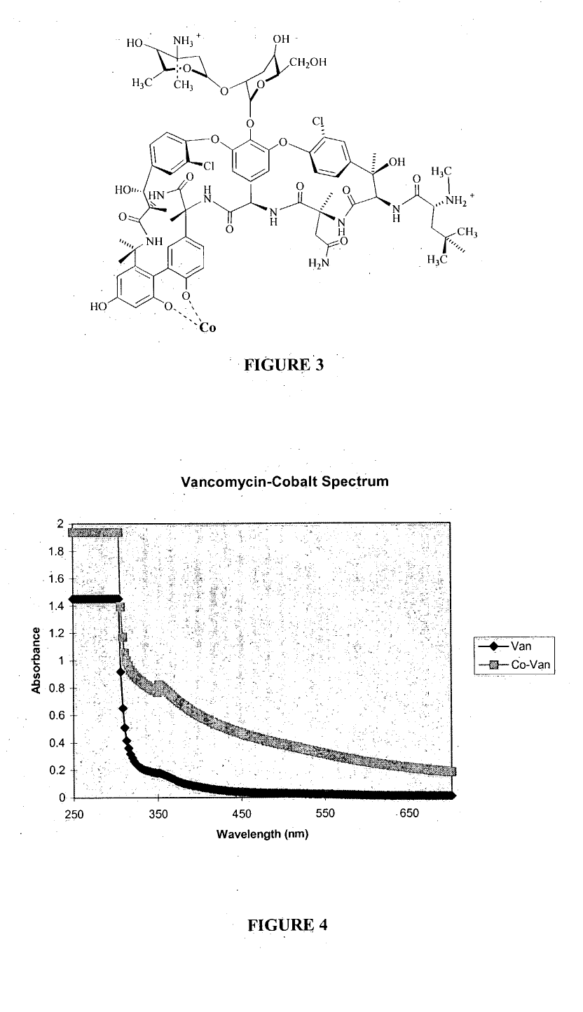

Redox-active metal complexes of vancomycin have been prepared, for example, the copper-vancomycin complex was disclosed as a useful adsorbent for resolution of racemic compounds, and its binding site was characterized by X-ray diffraction analysis, (shown in FIG. 2). See, Nair et al., Chirality 8:590-595 (1996). FIG. 3 illustrates a predicted structure of the cobalt-vancomycin complex, showing the cobalt interaction with the proximal phenolic hydroxyl groups on residues 5 and 6.

The glycopeptide antibiotics are a large class of substances either produced by microorganisms, or produced by microorganisms and thereafter subsequently modified in part. Two of these, vancomycin and teicoplanin, are commercially available at present, and many other examples are in clinical development. The entire class of glycopeptide antibiotics is described in Glycopeptide Antibiotics, edited by Ramakrishnan Nagarajan (Marcel Dekker, Inc., New York, 1994). Glycopeptide antibiotics that may be used in the chelated complex of the invention include, by way of example and not limitation, actinoidin, avoparcin, balhimycins, chloroorienticins, daptomycin, ereomycin, galacardin, helevecardin, orienticins, ristocetins, ristomycin A, teicoplanin, vancomycin, and derivatives thereof. A particularly preferred glycopeptide antibiotic is vancomycin. These glycopeptide antibiotics are also known as A82846A (ereomomycin), A82846B (chloroorienticin A), A 82846C (orienticin C). Additional examples of glycopeptide antibiotics are discussed below.

Galacardins (e.g., galacardin A and galacardin B) (Sankyo (Japan) are known by the chemical name: avoparcin alpha, 49-chloro-4B,50-di-O-alpha-D-mannopyranosyl-(CAS) and CAS REG NO: 137801-55-9. 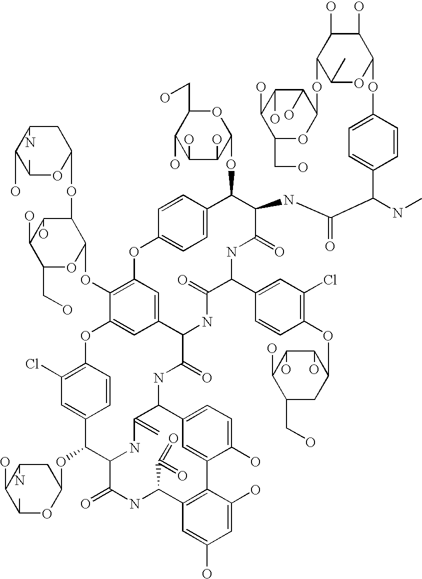
Galacardin A is produced from Saccharothrix sp. SANK 64289. In mice infected with Staphylococcus aureus 56, the in vivo efficacy (ED₅₀) of galacardin A was 19.0 mg/kg. The following MICs were reported (microgram/ml); Staph. aureus FDA 20P-3.12; S. aureus Sank 70175-6.25; Mycobacterium smegmatis ATCC 607-25.0 and Enterococcus faecalis subsp. Liquifaciens S-299-6.25. Galacardin B, is related compound (J. Antibiot., 1992, 45:297).

Pharmaprojects No. PA-42867-AA (Shionogi), is one of a series of antibiotics isolated from Nocardia orientalis PA-42867 (Ferm BP-1230). PA-42867 is one of a series of orienticins (Jpn. Kokai 87-174099) having potent activity against gram-positive bacteria, including Staphylococcus aureus SR14 (MIC of 0.78 microgram/ml) and in vitro activity against S. aureus SR2030 in mice, with an s.c. ED₅₀ of 2.31 mg/kg (Drug Data Rep. 1988 10:232; J. Antibiot., 1988 41:819 and 1506). The structure of PA-42867 is shown below:

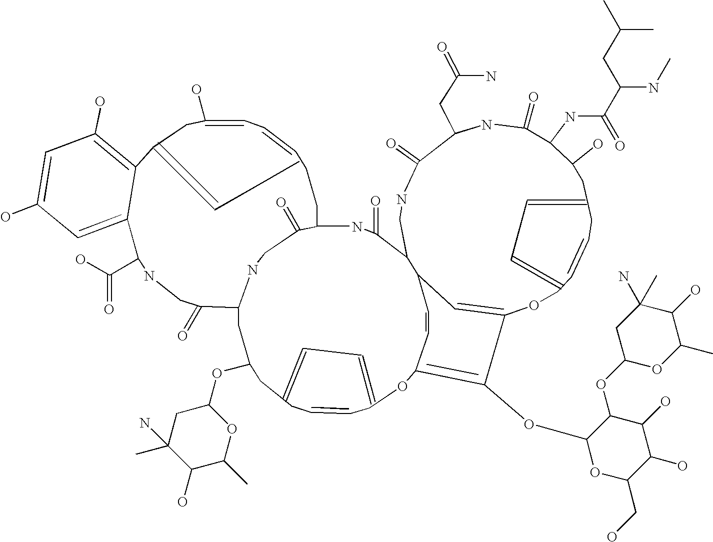

Pharmaprojects No. A-42867 (Biosearch Italia (Italy)), is a glycopeptide antibiotic isolated from a strain of Nocardia nov. sp. ATCC 53492. A-42867 is active against gram-positive bacteria and has similar in vitro activity to vancomycin and teicoplanin (qv). In mice infected with Streptococcus pyogenes, the ED₅₀ was 1.54 mg/kg (J. Antibiot., 1989, 42:497). This pseudo-glycone derivative is described in EP 0326029 to Riva et al.

Compounds such as Pharmaprojects No. A82846 (Eli Lilly (USA)), including A82846A, A82846C, chloroorienticin A, ereomycin, and Pharmaprojects Nos. LY-264826 and MM-45289, and further glycopeptide derivatives, including those with activity against vancomycin-resistant isolates, are described in EP 280570 and W09630401. A82846 has the chemical name, vancomycin,22-O-(3-amino-2,3,6-trideoxy-3-C-methyl-alpha-L-arabino-hexopyranosyl), (4″R)-(CAS), and is shown below:

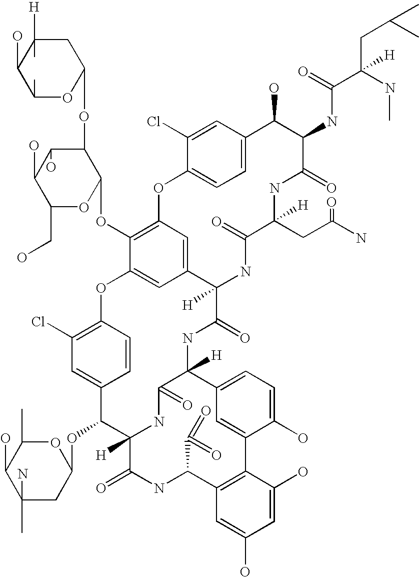

Pharmaprojects No. LY-307599 (Eli Lilly (USA)), also known as LY-191145, is one of a series of semisynthetic glycopeptides derived by modification of the glycopeptide antibiotic LY-264826 (qv), developed for the treatment of vancomycin-resistant enterococcal infections (Antimicrob. Agents Chemother. 39:2585 (1995)). The series of glycopeptides is also effective against other gram-positive pathogens and includes Pharmaprojects Nos. LY-309174, LY-309360 and LY-191145.

Pharmaprojects No. MDL-63166 (Biosearch Italia (Italy)), having the chemical name: (3S,6R,22R,23 S,26S,36R,38aR)-3-benzyl-10,19-dichloro-22,28,30,32,44-pentahydroxy-6-(D-lysylamino)-2,5,24,38,39-pentaoxo-2,3,4,5,6,7,23,24,25,26,36,37,38,38a-tetradecahydro-1H,22H-8, 11:18,21-dietheno-23,36-(iminomethano)-13,16:31,35-dimetheno(1,6,9)oxadiaza-cyclohexadecino(4,5-m)(10,2,16)benzoxadiazacyclotetracosine-26-carboxylic acid methyl ester, is a synthetic glycopeptide antibiotic derived from the methyl ester of deglucoteicoplanin. The structure of this glycopeptide is shown below:

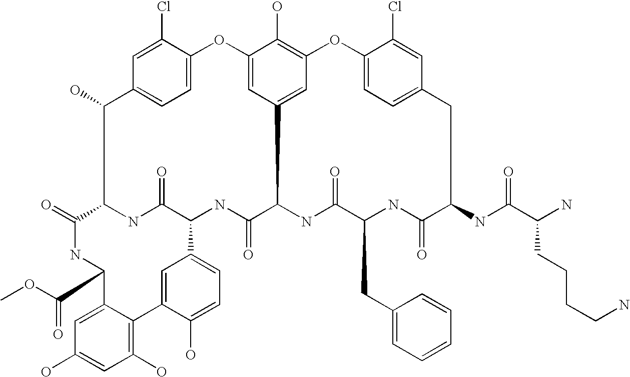

Pharmaprojects No. MDL-63246 (Biosearch Italia (Italy)) is a semisynthetic glycopeptide antibiotic, also known as RA-A-1, chemical name ristomycin A aglycone,5,3 1-dichloro-38-de(methoxycarbonyl)-7-demethyl-19-deoxy-56-O-(2-deoxy-2-((10-methyl-1-oxoundecyl)amino)beta-D-glucopyranosyl)-38-(((3(dimethylamino)propyl)amino)carbonyl)-42-O-alpha-D-mannopyranosyl-N15-methyl-(CAS), CAS REG NO: 148868-06-8. The structure of this compound is shown below:

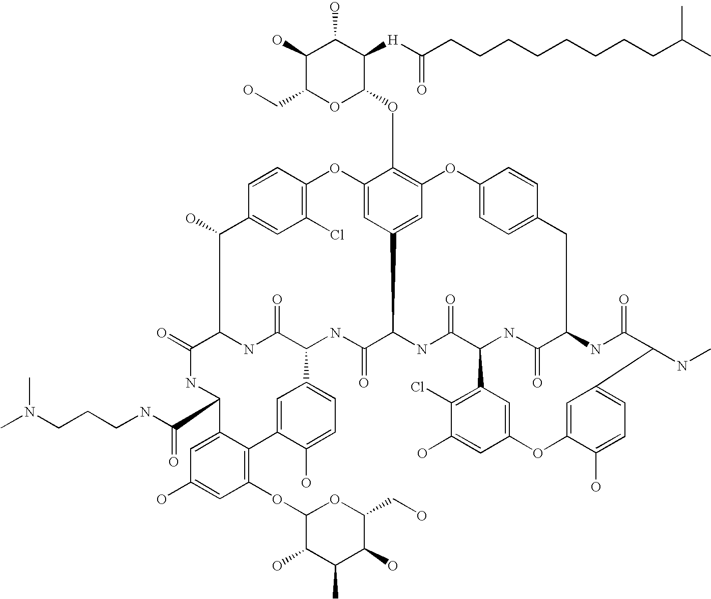

Balhimycin derivatives are described in EP 521408. These compounds include balhimycin R, balhimycin V, des-gluco-balhimycin, des-methyl-des-gluco-balhimycin, des-methyleucyl-balhimycin, methyl-balhimycin, ureido-balhimycin and des-methyl-balhimycin, the most active of which is des-methyl-balhimycin, which was reported as having the following MICs (microgram/ml): Staphylococcus aureus SG511-0.1; S. aureus 285-0.1; S. aureus 503-0.05; Streptococcus pyogenes 77A-0.05; Strep. pyogenes 308A-0.5; Strep. falcium D-0.2; Escherichia coli-10. The structure of this compound is shown below:

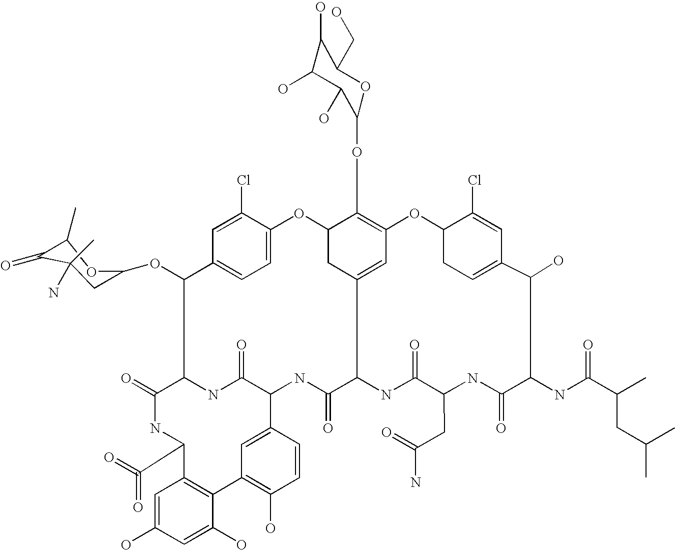

Pharmaprojects No. MM 55268, and related compound MM 55266 (GlaxoSmithKline (UK) (J. Antibiot. 44:807 (1991)), is known as ristomycin A aglycone, 5,22,31,45,55-pentachloro-7-demethyl-64-O-demethyl-34-O-(2-deoxy-2-((1-oxodecyl)amino)-alpha-D-glucopyranuronosyl)amino)-44-O-beta-D-glucopyranosyl-56-O-.beta.-D-mannopyranosyl-N15-methyl-(CAS), CAS REG NO:137053-19-1. MM 55268 is a glycopeptide antibiotic isolated from Amycolatopsis sp. NCIB and shows activity against gram-positive bacteria, with a similar activity to vancomycin. MICs (microgram/ml) were observed as follows: Bacillus subtilis ATCC 6633-2 (cf 0.25 for vancomycin); Micrococcus luteus-2 (cf 2); Staphylococcus aureus Oxford-4 (cf 2); Staph. aureus V573 MR-1 (cf 2); Streptococcus pyogenes CN10-0.25 (cf 1) and Strep. faecalis I-4 (cf 2). The structure of this compound is

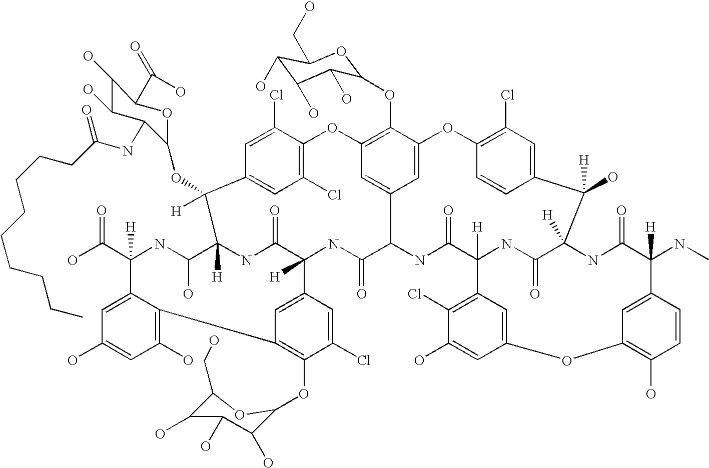

Pharmaprojects No. S-3662 (Shionogi (Japan)) is a glycopeptide antibiotic related to vancomycin for parenteral treatment of gram-positive bacteria, and reported to be is superior to vancomycin against laboratory strains of aerobic and anaerobic gram-positive bacteria. In systemic infections in mice, S-3662 had the following ED₅₀s (mg/kg): Staphylococcus aureus (2.63); Streptococcus pyogenes (0.49); Strep pneumoniae (0.39) and Enterobacter faecalis (0.95). S-3662 also showed therapeutic efficacy against subcutaneous and urinary tract infections. The structure of this compound is shown below:

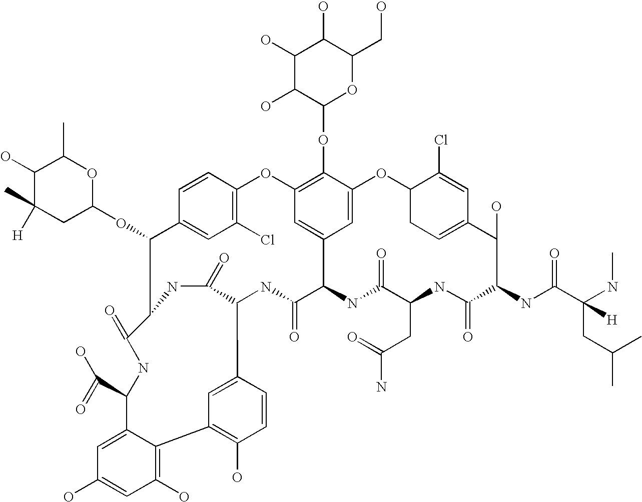

Pharmaprojects No. MM-49721, was isolated from Amycolatopsis orientalis (GlaxoSmithKline (UK)), and is known by the chemical name vancomycin,22-O-(3-amino-2,3,6-trideoxy-3-C-methyl-alpha-D-arabino-hexopyranosyl)-2′-O-de(3-amino-2,3,6-trideoxy-3-C-methyl-alpha-L-lyxoyranosyl)-10,19-didechloro-2′-O-(6-deoxy-alpha-D-talopyranosyl)-(CAS), CAS REG NO:126985-52-2. MM-49721 possesses good activity against gram-positive bacteria, and has the following MICs (microgram/ml): Bacillus subtilis-2; Staphylococcus aureus-1; Streptococcus pyogenes-1 and Streptococcus faecalis-1 (J. Antibiot. 43:931 (1990)). The structure of this compound is shown below:

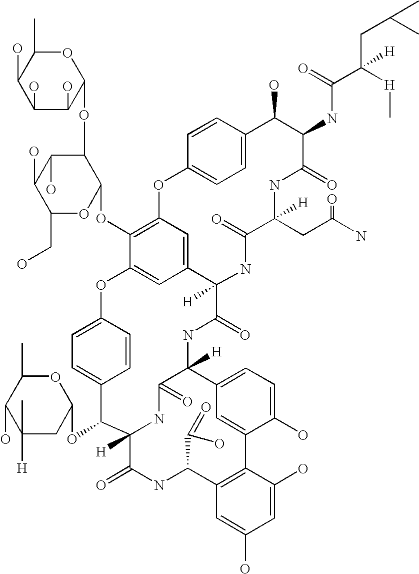

Pharmaprojects No. UK-68597 (Pfizer (USA)) is a glycopeptide antibiotic, which was isolated from Actinoplanes sp. ATCC 53533 and reported at the 29^{th} ICAAC (Houston), 1989, Abs. 415-416). Daptomycin and daptomycin derivatives, such as the prodrug, 4-(phenyl)benzyl substituted ornithine daptomycin (Cubist); Pharmaprojects No. AC-986446, a semisynthetic mannopeptimycin glycopeptide, related to the natural product AC-98; and Compound F, a lipidated glycopeptide for the treatment of gram-positive infections, are examples of some newer glycopeptide antibiotics still in the early experimental stages.

U.S. Pat. No. 5,977,062 to Cooper describes additional members of the glycopeptide group of antibiotics. These compounds are derivatives of known glycopeptide antibiotics that include vancomycin (U.S. Pat. No. 3,067,099 to McCormick et al); A82846A, A82846B, and A82846C (U.S. Pat. No. 5,312,738 to Hamill et al.); PA-42867-A and PA-42867-B (U.S. Pat. No. 4,946,941 to Kondo et al.); A83850 (U.S. Pat. No. 5,187,082 to Hamill et al.); A-4696 pseudo-aglycone or avoparcin (U.S. Pat. No. 4,322,343 to Debono); actinoidin, also known as K288 (J. Antibiotics Series A 14:141 (1961)); helevecardin (Chem. Abstracts 110:17188 (1989)); galacardin (Chem. Abstracts 10:17188 (1989)); and M47767 (EP 0339982 to Athalye et al). Some of these compounds are discussed above.

Many derivatives of naturally occurring glycopeptides have been made. For example, N-alkyl and N-acyl derivatives of the glycopeptides vancomycin, A51568A, A51568B, M43A and M43D have been prepared, described in U.S. Pat. Nos. 4,639,433, 4,643,987, and 4,698,327 to Bender, U.S. Pat. No. 6,455,669 to Judice, and U.S. Pat. No. 5,998,581 to Berglund. Several of these compounds were reported to exhibit activity against vancomycin-resistant isolates. See Nicas et al., Antimicrobial Agents and Chemotherapy, 33(9):1477-1481 (1989). In addition, EP 0435503 to Nagarajan et al., describes certain N-alkyl and N-acyl derivatives of the A82846 glycopeptides, factors A, B, and C. EP 0435503 and EP 0667353 to Cooper et al. also describe reductive alkylation of a variety of glycopeptides including vancomycin, A82846A, A82846B, A82846C, and orienticin A. These references describe the introduction of a great variety of alkyl or alkenyl groups into the parent glycopeptide. Therefore, various derivatives (including analogs) of the glycopeptide antibiotics are readily identifiable and synthetically accessible, and are therefore encompassed within the present invention.

B. Ribonucleoside Antibiotics:

Antibiotics of the ribonucleoside class may also be used in the present invention. Antibiotics of the ribonucleoside class that may be used are lincosamides, such as lincomycin and clindamycin, and derivatives thereof. Other antibiotics in this class that are water-soluble and possess potential metal ligands such as S, O and N are also potential candidates.

Various celestosaminide derivatives related to lincomycin can be prepared by adding different organic acids to culture broths of Streptomyces caelestis. One of these compounds, desalicetin-2′-(4-amino salicylate), has potent antibacterial activity in vitro.

Pharmaprojects No. BU-2545 (7-O-methyl-4′-de-n-propyllincomycin) is a macrolide antibiotic structurally related to lincomycin, and is isolated from Streptomyces sp H230-5. BU-2545 exhibits antibacterial activity against anaerobes and gram-positive aerobes (J. Antibiot., 33:751 (1980) and 34:596 (1981)). The structure of this compound is shown below:

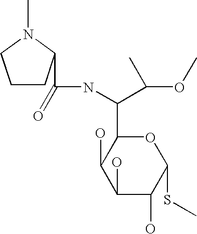

Therefore, there are additional derivatives and variants of the ribonucleoside antibiotics that are useful in the antibiotic-metal complexes described herein and the methods of the invention.

C. Quinolone Antibiotics:

The chelated complexes of the present invention may also be applicable to other classes of antibiotics, such as the quinolones. The quinolones include, but are not limited to cinoxacin, ciprofloxacin, fleroxacin, gatifloxacin, levofloxacin, lomefloxacin, moxifloxacin, nalidixic acid, norfloxacin, ofloxacin, perfloxacin, sparfloxacin, trovafloxacin, and derivatives thereof. Nalidixic acid is a preferred quinolone antibiotic.

Sparfloxacin is the first among a new generation of fluorinated quinolones, which has demonstrated efficacy in the treatment of community-acquired pneumonia, and Acute Bacterial Exacerbations of Chronic Bronchitis, caused by susceptible strains of Chlamydia pneumoniae, Mycoplasma pneumoniae, Enterobacter cloacae, Haemophilus influenzae, Haemophilus parainfluenzae, Klebsiella pneumoniae, Moraxella catarrhalis, Staphylococcus aureus, or Streptococcus pneumoniae. In addition, sparfloxacin is highly active in vitro against multi-drug resistant strains of the gram-negative pathogens Haemophilus influenzae and Moraxella catarrhalis. Additionally, it has demonstrated activity against the atypical pathogens (such as Mycoplasma pneumoniae, Legionella pneumophilia and Chlamydia pneumoniae). Sparfloxacin exerts its antibacterial activity by inhibiting DNA gyrase, an enzyme which assists in DNA replication, deactivation and transcription.

Synercid® (quinupristin/dalfopristin), for the treatment of vancomycin resistant infections, is a novel, injectable streptogramin antibiotic made of two molecules, quinupristin and dalfopristin. In combination, they create a synergistic bactericidal agent that kills bacteria by inhibiting protein synthesis. In laboratory tests, Synercid has been shown to be active against the major gram-positive strains of Enterococcus faecium, Staphylococcus aureus, Staphylococcus epidermis and Streptococcus pneumoniae, including multidrug-resistant strains.

III. Metals of the Antibiotic-Metal Complex:

The preferred metals of the present invention include the transition metals and the lanthanides. The transition metals are particularly preferred because of their high oxidation-reduction activity in neutral aqueous media. It is likely that these metals catalyze the oxidation of chemiluminescent substrates, such as the oxidation of luminol by hydrogen peroxide, as described by Rost et al., J. Biolumin. Chemilumin. 13:355-464 (1998)). The antibiotic-metal complexes can directly catalyze peroxide-driven chemiluminescent reactions (for example, reactions involving luminol, its aromatic derivatives, lucigenin, penicillin, luciferin and other polyaromatic phthalylhydrazides) without the use of an enzyme catalyst such as horseradish peroxidase or microperoxidase. Additional compounds have been found to provide chemiluminescence, as described further in U.S. Pat. No. 6,451,536 to Fodor et al, such as 2,3-dihydro-1,-4-phthalazinedione; the 2,4,5-triphenylimidazoles, (e.g., lophine), including the para-dimethylamino and -methoxy substituents; and certain oxalates, generally oxalyl active esters, e.g., p-nitrophenyl.

Factors influencing the catalytic efficiency of individual metals include pH, ionic strength and oxidation state. Chelation chemistries that would alter the oxidation state or steric availability of the metals during catalysis could also influence the optimum catalytic activity as sensed by the time dependent emission of photons. The transition metals, cobalt, copper and chromium are preferred metal complexes because of their inherently high catalytic efficiency for the peroxide-driven oxidation of luminol, as described by Rost et al. (ibid). However, other redox-active metals can be as efficient or more than the aforementioned metals.

Exemplary transition metal include zinc (Zn), copper (Cu), nickel (Ni), cobalt (Co), iron (Fe), manganese (Mn), chromium (Cr), and technicium (Tc), as well as their isotopes. Cobalt, iron, manganese and chromium are particularly preferred as they yield the most catalytically active complexes on a molar basis. Cobalt and chromium complexes are especially preferred.

Exemplary lanthanide metals include europium (Eu), gadolinium (Gd), terbium (Tb), dysprosium (Dy), erbium (Er), lutetium (Lu), as well as their isotopes. Gadolinium complexes of these antibiotics can be used in NMR imaging in patients or on suspected infectious samples. A unique and useful aspect of terbium and europium complexes is that neither the metal salts nor the antibiotic are fluorescent; however, some of the chelates are fluorescent. For example, upon addition of the lanthanide salts, terbium or europium chloride, to solutions of polymyxin, a blue fluorescent emission can be observed at 400-450 nm when illuminated with 330 nm light, and is useful as an epifluorescence microscopy label for E. coli and Salmonella cells. Similarly, the glycopeptide, ribonucleoside and quinolone antibiotics can be complexed with lanthanides to produce fluorescent complexes having applications in imaging and detection of microorganisms.

IV. Methods of Antibiotic-Metal complex Preparation and Characterization:

Antibiotic-metal complexes are readily prepared in aqueous solution (e.g., an aqueous buffer), although non-aqueous solvents and/or mixed solvents can be used provided the metal salt and antibiotic are sufficiently soluble to form a chelated complex and bind to the target microorganism present in or on the sample. If using a buffer, volatile buffers, such as acetic acid, ammonium acetate, and ammonium bicarbonate are preferred.

In one embodiment of the invention, the chelated antibiotic-metal complex is synthesized by admixing (i) a water soluble salt of a metal selected from the group consisting of transition metals and lanthanides with (ii) an antibiotic selected from the group consisting of glycopeptide antibiotics, ribonucleoside antibiotics, and quinolone antibiotics, in (iii) a solvent for the metal salt and the antibiotic. The admixing is conducted under conditions effective to promote chelation of the metal by the antibiotic, thereby forming a solution of the chelated antibiotic-metal complex.

The method generally involves dissolving crystalline or powdered antibiotic to form a concentrated solution, preferably greater than 0.5 M, and adding water soluble metal salts to provide a slight molar excess over the antibiotic. If the complex formation is performed at lower concentrations of antibiotic, the complex of course can be dried or concentrated. Chelates formed in solution can be isolated by separating the free metal from the antibiotic. The resulting chelates may also be desalted, for example by dialysis or gel filtration (e.g., dialysis in narrow-pore molecular weight cut-off tubing for example, from Spectro-Por, or by gel filtration on GPC media such as Sephadex G-25). The purified antibiotic-metal complex can be also dried if desired, preferably by freeze drying or, alternatively, by spray drying. Other suitable solvents, separation, desalting and drying techniques can be used, as are well known in the art.

Optionally, metal chelating ligands can be added to the antibiotic. Reductive alkylation with aromatic carboxaldehydes, the monocarboxaldehyde of 2,2′-dipyridine, salicylaldehyde or protocatechualdehyde, for example, can be used to add a suitable metal binding cavity to the antibiotic molecule to chelate several transition metals such as copper, nickel, zinc, technetium, and preferably cobalt, iron, manganese, or chromium. The aforementioned ligands, including 2,2-dipyridyl monocarboxlic acid, salicylic acid, and protocatechuic acid, could alternatively be grafted onto the antibiotic through an amide linkage as preformed, isolated N-hydroxysuccinimide esters. The ligands could either be used preloaded with the metals as reactive chelates, or optionally, chelated after the conjugates are formed. The method of forming a complex with an antibiotic modified to contain additional metal chelating ligand is similar to the procedure described above.

Antibiotic-metal complexes, once formed, can be further characterized by standard analytic techniques, such as combustion analysis, NMR, EPR, and electronic spectroscopy, for example. These procedures can also be accompanied by a bio-assay method to ensure preservation of bacterial binding activity, and/or anti-microbial activity. A typical bio-assay can be conducted as follows. Bacteria, diluted to a cell concentrations of 1-100 CFU/mL (colony forming unit/mL), preferably 10 CFU/mL, are treated with an antibiotic-metal complex (e.g. vancomycin-Co (II) complex (as described in Example 1) at 0.1-1000 μg/mL, preferably 1-100 μg/mL, and most preferably 30 μg/mL, at room temperature for a time sufficient to achieve binding (usually 5-60 minutes). The cells are removed, by for example, centrifugation, magnetic microbeads, or other method of pathogen capture, washed and resuspended in assay solution. Chemiluminescence is measured after the addition of oxidizable substrate and peroxide (e.g., Luminol reagent purchased from NEN Life Sciences, Boston, Mass.), using a Luminator® luminometer). The minimum number of cells that are detectable using the chemiluminescent antibiotic-metal complexes is approximately 10-100 cells per sample.

Alternatively, an end-point determination for Minimum Inhibitory Concentration (MIC) of the antibiotic can be conducted according to standard microbiological procedures. MIC is determined by testing the ability of bacteria to grow in the presence of varying concentrations of an agent to be tested for anti-microbial activity. One variation of this procedure is performed as follows, with other variations of this general protocol being within the ability of one skilled in the art. A stock culture of ˜108 CFU/ml is used to inoculate a 5 ml portion of Trypticase Soy Broth, using a 0.1 ml aliquot. An antibiotic is added to the inoculated samples, at concentrations ranging from about 1 μg/ml to about 100 μg/ml, and the samples are grown for 24 hours in a 37° C. incubator. The sample turbidities are then compared with negative controls containing no antibiotic. The minimum concentration yielding no bacterial growth is the MIC.

V. Methods for Using the Antibiotic-Metal Complexes:

The complexes of the present invention are useful to bind to and detect gram-positive pathogenic bacteria or residues thereof, and, in some embodiments, to bind to and detect non-pathogenic microorganisms.

Accordingly, one embodiment of the invention relates to a method for conducting a chemiluminescent assay of microorganisms in a sample. The method involves contacting a sample suspected of containing the microorganism on interest, with the chelated complex on the invention, i.e., a chelated complex of an antibiotic (glycopeptide, ribonucleoside or quinolone antibiotic) and a detectable transition or lanthanide metal label. Complex-bound microorganisms are then separated from unbound complex. An oxidizable substrate and a source of peroxide are then added to the complex-bound microorganisms. The oxidizable substrate and peroxide source can be added to the complex-bound microorganisms individually or the oxidizable substrate and peroxide source an first be mixed together, and the mixture added to the complex-bound microorganisms. The complex-bound microorganisms are then detected by measuring luminescence. Luminescent light emission is typically detected using a photodetector.

The methods of the invention are not limited as to the sample that can be tested. Numerous fluid samples can be tested by these methods. These include drinking water; animal products, e.g., chicken and meat such as hamburger, intended for human consumption); and a variety of patient samples such as lung aspirate, blood, tissue samples, and stool samples. In addition, dry samples such as soil, can be added to an appropriate liquid medium, and similarly analyzed.

The oxidizable substrate may be chemiluminescent substrate, for example luminol, lucigenin, penicillin, luciferin, polyaromatic phthalylhydrazides, and derivatives thereof. The oxidizable substrate may also be any other substrate as are known in the art, such as those described in U.S. Pat. No. 6,451,536 to Fodor et al. The peroxide source can be the exogenous addition of hydrogen peroxide, benzoyl peroxide or cumyl peroxide, or may be an enzyme such as glucose oxidase or an amino acid oxidase.

Microorganisms present on or in the sample may be removed from the sample to be tested by washing or other physical methods for sample preparation. For example, the sample may be contacted using a swab and any microorganisms present on the swab can be suspended into aqueous buffer solution. The microorganisms on the surface or within the sample may also be washed off using buffer, disrupting the structure of the sample if necessary, by mincing or shredding the sample, for example. Alternatively, the microorganisms may be disassociated from the sample by sonicating the sample in buffer. Buffer solutions containing high salt, low or high pH, or additional solvents may also be used to disassociate the microorganisms from the sample to be tested.

In one preferred method, the sample may be treated first with the antibiotic and metal such that any microorganisms present become labeled with the antibiotic-metal complex. Such in situ labeling can be performed prior to the removal of microorganisms from the sample. Alternatively, once the microorganisms are removed from the sample, the microorganisms may be labeled with the antibiotic-metal complex.

The microorganisms isolated from the sample may be concentrated by centrifugation, filtration or drying. Alternatively, adsorptive particles (e.g. magnetic immuno-microbeads or phage-microbeads) may be used to concentrate the sample containing microorganisms. Adsorptive particles are microbeads are typically made of polystyrene, latex, polymer coated ferrite, polymer coated super-paramagnetic materials, polymer coated and uncoated magnetic materials, silica, and cross-linked polysaccharides. Preferred microbeads are non-porous monodisperse superparamagnetic particles comprising polystyrene and divinyl benzene with a magnetite core (8±2×10⁻³ cgs units) and a diameter of about 2-5 μm. Microbeads with reactive groups on their surfaces (e.g., SH, OH, NH₂, COOH, tosyl, etc.) are commercially available. These microbeads can be used for covalent attachment of numerous protein or nucleic acid ligands. For example, beads which have streptavidin attached can be used to bind a component from a sample that is attached to biotin.

Similarly, antibodies or antibody fragments (e.g., Fab) specific for one or more target microorganisms can be attached to magnetic microbeads via the reactive groups in order to facilitate isolation and concentration of the microorganisms for quantitative or qualitative testing. The target microorganism can be isolated using specific antibodies attached to microbeads and the antibiotic-metal complex is allowed to bind to the pathogen either before or after isolation of the microorganisms. For example, rapid capture of Listeria cells in a complex sample can be effected using anti-Listeria antibody on magnetic microbeads. Use of this immuno-microbead method requires obtaining or preparing antibodies specific for Listeria and attaching them to the reactive groups on the microbeads. Antibodies with a broader range of specificities to target pathogens (e.g., antibodies that bind to peptidoglycan or lipopolysaccharide) can also be attached to microbeads, and antibiotic-metal complexes of more or less desired specificity can be used as a probe for particular species.

One method of isolating target bacterial microorganisms involves using microbeads having attached bacteriophage, phage ghosts or purified phage sheath proteins. The selective binding function of the phage or purified phage sheath proteins is preferable to using antibodies because phage for particular target species are readily available and can be very specific. A bacteriophage specific for Listeria could be attached to microbeads. For example, B1 Phage of Listeria monocytogenes (ATCC 23074), is commercially available, and when attached to microbeads, can be used as a Listeria specific reagent. One could also inactivate purified phage particles using hypo-osmotic shock, rapidly dilution into distilled water, or by brief exposure to low pH, causing the loss of phage DNA. The phage “ghosts” are then immobilized on activated magnetic particles (e.g. tosyl-activated particles) in much the same manner as antibodies are immobilized.

Alternately, a phage sheath protein carrying the recognition site for the cell walls of interest (e.g., a Listeria species) is purified from intact phage particles and attached to microbeads. A phage sheath protein can also be genetically engineered as a fusion peptide comprising a nickel-binding site to permit affinity purification from crude E. coli lysates once cloned into the appropriate expression vector. A similar approach preparing fusion proteins containing the endolysin protein is described in Loessner, et al., Appl. and Environm. Microbiol. 62:3057-3060 (1996). Using either purified phage sheath protein, phage ghosts, or fusion proteins comprising the sequence for phage sheath protein, the phage is then attached to microbeads and used as a target microorganism specific capture agent. The bacterial microorganisms, in this example Listeria, can be treated with the antibiotic-metal complex either before isolation or after isolation from the sample being tested. Detection and quantitation is effected in both cases by the chemiluminescence of the antibiotic-metal complex in the presence of an oxidizable substrate and a source of peroxide.

Similarly, bacteriophages specific for other organisms can be used as specific reagents for isolating the microorganisms for ease of quantitative and qualitative analysis. For example, Staphylococcus aureus (subsp. aureus Rosenbach) can be specifically targeted using bacteriophage P1 (ATCC 11987). The range of bacteriophages available and the bacteria for which they are specific will be realized to be vast by those skilled in the art. For example a list of phage types is available from the American Type Culture Collection (ATCC). Other such depositories also publish equivalent data in their catalogues and this may be used to identify possible phage “reagents” for use in the present method. Phages may be used, inter alia, in aqueous suspension or in freeze dried form e.g. on microtiter plate wells. In this manner plate luminometry can be used.

In addition to phages obtained from a depository, an additional source of phages can be provided by isolating them from suitable environments, such as the environment where the target bacteria are themselves to be found. For example, it is possible to isolate phages specific to both Campylobacter spp. and Salmonella spp. from effluent from a poultry processing plant. Isolation techniques will be well known to those skilled in the art and are exemplified, for example, by Loessner et al., Appl. and Environm. Microbiol. 56:1912-1918 (1990), and Adams, “Bacteriophages” (1959), Pub. Interscience Inc. pp 447-455. Isolation of additional bacteriophages that can be used in the present methods is described in U.S. Pat. No. 6,322,783 to Takahashi.

The range of media available for selective promotion of growth of a particular bacterial type will also be known to those skilled in the art and these may function by positive action or by e.g. inhibition of other organisms. Examples of such media are illustrated by reference to supplier's manuals, e.g. such as those available from UNIPATH® Limited, Wade Road, basingstoke, HANTS, RG24 OPW, UK “Selective Microbiology for Food and Dairy Laboratories”, or e.g. the OXOID® manual. These publications list, for example, media capable of favoring growth of Campylobacter, Listeria and Yersinia. Similarly methods for isolation of food pathogens for preparation of test samples are well known. Additional useful references are the microbiology manuals: Bergey's Manual of Systematic Bacterial Classification and the DIFCO manual.

Numerous methods are known in the art for covalently attaching chemical moieties to surfaces, for example the microbeads described above. Any of the art-recognized methods can be used, for example, cross-linking reagents, chemical derivatization methods, etc. to attach intact phage, phage ghosts or phage proteins to microbeads or other capture agent. Alternatively, antibodies or antibody fragments specific for the phage can be attached to the surface of the microbead, and used to bind pathogens from a sample, when phage has been added to the sample to bind the pathogen with high binding specificity. As will be appreciated, other variations are also possible, and are encompassed within the disclosed method of utilizing phage for specific capture of microorganisms.

In some instances, it may be desirable to test a biological sample in a more invasive manner to test for intracellular pathogens or adherent pathogens. Intracellular pathogenic microorganisms include such organisms as parasites (e.g., Rickettsia, Chlamydia, Plasmodia), viruses (e.g., viral genes or expression products), or aberrant proteins associated with a pathological condition (e.g., prions). Adherent pathogens are pathogens that bind strongly to host tissue, for example, using pili, and may not be removed by washing. Such biological samples may be treated to generate a cellular suspension, such as by homogenizing the tissue, or may even be disrupted so that cellular contents are released. Intracellular pathogens or pathogens present in cell suspensions may be captured and detected using antibody or phage attached to microbeads. Alternatively, these pathogens may be detected using a chemiluminescent agglutination assay, as described in detail below.

Finally, once microorganisms have been removed from the sample by phage or antibody binding, the number of microorganisms present is determined by measuring the luminescence in the presence of an oxidizable substrate (e.g., luminol) and a source of peroxide.

Preferred separation methods for target microorganisms include immuno-sedimentation using either magnetically accumulated microbeads or gravity sedimentation. Filtration of bacteria or fungi from buffer solution can also be performed. Several methods for isolation of microorganisms from food and water have been published, e.g., Fratamico Food Microbiol. 9:105-113 (1992), and Pyle Appl. Environm. Microbiol. 65:1966-1972 (1999)). Use of these immuno-sedimentation techniques provide several advantages over the aforementioned alternative selective methods such as speed, simplicity, minimization of handling, and elimination of the need for incubation equipment.

These are just a few examples of how the antibiotic-metal complexes can be used in assays to detect the presence of microorganisms. Once skilled in the art can envision numerous permutations using in situ labeling, various methods of removing unbound label, augmenting the sensitivity with antibodies or phage particles and the like in order to tailor the assay for the desired detection.

Potential gram-positive pathogenic targets for the antibiotic-metal complex of the invention include, but are not limited to, aerobic spore-forming Bacilli (e.g., Bacillus anthracis, B. subtilis, B. megaterium, and B. cereus), anaerobic spore-forming Bacilli (e.g., Clostridium botulinum, whose exotoxins cause botulism, C. tetani, C. perfringens, whose exotoxins cause tetanus, C. novyi, C. septicum, C. histolyticum, C. tertium, C. bifermentans, and Clostridium sporogenes), Listeria (e,g, Listeria monocytogenes), Nocardia (e.g., Nocardia asteroids and N. brasiliensis, Pneumococci (e.g., (Diplococcus pneumoniae), Staphylococci (e.g., Staphylococcus aureus, S. epidermidis, and S. albus), and Streptococci (e.g., Streptococcus pyogenes, S. pneumoniae and S. salivarus).

Other potential bacterial targets for the antibiotic-metal complex of the invention include, but are not limited to, Bacillus anthracis (anthrax), and Mycobacteria species such as Mycobacterium tuberculosis hominis, M. bovis, M. avium, M. paratuberculosis, and M. leprae.

The complexes are also capable of binding to and detecting permeabilized gram-negative bacterial cells. The outer membrane of gram-negative bacteria may not allow access to binding sites of the antibiotics encompassed in the methods of the invention, thus preventing the antibiotics from binding to their target sites. However, the gram-negative bacterial cells may be permeabilized by treating the cells with a chelating agent (e.g., EDTA) destabilizing the structure of the outer membrane lipopolysaccharide (LPS) layer, with a corresponding increase in cell permeability, or by treating the cells with solutions of high pH. Another method of permeabilizing gram-negative cells is by pre-treating the cells with the non-toxic fragment of polymyxin B, polymyxin B nonapeptide, which renders gram-negative bacteria susceptible to substances known to be unable to pass through the outer membrane envelope. These permeabilization methods are an effective method of rendering the gram-negative bacterial cell membrane susceptible to detection using the antibiotic-metal complexes described herein. Permeabilized gram-negative bacteria that may be targeted by the present antibiotic-metal complexes include, but are not limited to, Neisseria (e.g., Neisseria menigitidis and N. gonorrhoeae), Flavobacter and Salmonella (e.g, Salmonella typhosa, S. typhimurium, S. derby, S. choleraesuis, S. enteritidis and S. pullorum), as well as other Enterobacteriaceae (e.g, the Coliform bacteria such as Escherichia coli, Aerobacter aerogenes, Klebsiella pneumoniae; the Shigellae such as Shigella dysenteriae, S. schmitzii, S. arabinotarda, S.flexneri, S. boydii, Shigella sonnei; and other enteric bacilli such as the Proteus species including Proteus vulgaris, P. mirabilis, and P. morgani) and all other classes of aerobic and anaerobic gram-negative microorganisms, that have been permeabilized such that the antibiotic-metal complexes of the present invention are permitted access to the cell membrane.

Additional suitable target microorganisms include protozoans such as Plasmodia, which can be targeted using the ribonucleoside antibiotics, preferably a lincosamide. Use of the antibiotic-metal complexes of the invention allow for rapid detection and diagnosis of disease in a patient suffering from malaria. The rapid detection and diagnosis using the lincosamides-metal complex provides a significant improvement over the current methods for diagnosis, which require preparation of blood smears and skilled interpretation. Epifluorescence using a lanthanide as the metal chelate or chemiluminescence using a transition metal chelate can also be used in conjunction with traditional methods of diagnosis of malaria, for example, concurrent analysis of microscope slides containing blood smears from a patient.

An exemplary embodiment of a simple binding assay that can be used with the chelated complexes of the invention involves labeling gram-positive cells in suspension, pelleting the cells by centrifugation or isolating the cells by filtration or immuno-separation, washing unbound label, and detecting the bound complexes with chemiluminescent reagents. Bacterial cells are diluted from stock cultures and the cell suspensions are labeled at room temperature with a antibiotic-metal complex at a concentration sufficient to achieve labeling. Generally a concentration of the antibiotic-metal complex of about 0.01-0.05 mg/mL is sufficient. The labeled cells can, optionally, be collected by centrifugation, filtration on micro-porous filters of the polycarbonate film type (Osmonics, Inc.) or rapid immuno-separation using antibody coated super para-magnetic particles. Phage coated paramagnetic particles may also be used. The labeled cells are then washed and resuspended in peptone water for assay with preferably, hydrogen peroxide/luminol or any number of oxidizable chemiluminescent substrates, including lucigenin, penicillin and the like.

Another exemplary embodiment of an assay that can be used with the chelated complexes of the invention is for the detection of bovine tuberculosis, which is caused by Mycobacterial infection of cattle. Current analytical methods for the detection of bovine tuberculosis require a sixteen week period for a diagnosis. Using the chelated complexes described herein, the presence of this disease can be determined in a few hours using the following procedure. A fecal or milk sample can be conveniently screened for the presence of Mycobacteria by filtering the milk or fecal matter (suspended in buffer, e.g., phosphate buffered saline) through a 5 μm filter, which captures the clumps of waxy Mycobacteria cells. Detection is then easily accomplished by labeling with an antibiotic-metal complex, for example, a glycopeptide antibiotic-metal complex such as a quinolone-Co complex (e.g., ciprofloxacin-Co complex), and detecting chemiluminescence in the presence of an oxidizable substrate and a source of peroxide. Alternatively, the Mycobacteria cells could be captured from the sample using a phage attached to microbeads which is specific for Mycobacteria, and then the cells could be detected by labeling with an antibiotic-metal complex and using chemiluminescence.

The binding affinity of a chelated complex to a specific microorganism is related to the binding affinity of the uncomplexed antibiotic for that organism. Therefore, in certain embodiments, it may be preferred to use a broad spectrum antibiotic such as vancomycin, and thus provide targeting and detection of a broad spectrum of microorganisms of interest. In other embodiments, it may be desirable to target a specific species or family of microorganisms. The targeting specificity can be provided through the correct choice of the antibiotic, or can be provided by means of antibodies or phage particles specific for a particular organism, as will be discussed further below.

For example, the ribonucleoside antibiotics are useful as probes for, inter alia, pneuomococci, S. pyogenes, and viridans streptococci, B. fragilis, Bacteroides melaninogenicus, Fusobacterium, Peptostreptococus, Peptococcus, Clostridium perfringens, Actinomyces israelii and Nocardia asteroides, Pneuomcystis carinii, and T. gondii. The ribonucleoside antibiotics have also shown efficacy against protozoans such as Plasmodium falciparum and Plasmodium vivax. The anti-microbial spectrum of this class of antibiotics is narrower than the beta lactam antibiotics or the cephalosporins, and therefore may be useful as more selective probes for potential pathogens such as Streptococcus pyrogenes or Staphylococcus aureus, and may have useful clinical applications in screening samples for potential sources of sepsis or other important nosocomial infections.

The antibiotic-metal complexes of the invention can also be used as magnetic resonance imaging agents. Paramagnetic metals alter the magnetic field in their vicinity such that paramagnetic metals can be easily imaged within a patient's body using magnetic resonance imaging. By including a paramagnetic metal in the antibiotic-metal complex, the metals can be targeted to the location of a site of infection within the body of a patient. Gadolinium is a preferred metal useful for magnetic resonance imaging because of its extremely high nuclear spin, which produces a very strong perturbation in the homogeneity of an applied magnetic field. For imaging the presence of pathogenic bacteria, for example, a Gd-antibiotic chelated complex could be utilized with an antibiotic specific for a particular pathogen.

Alternatively, by forming the antibiotic-metal complex with a radioactive metal, preferably one having a short half-life, the complex can be used as an agent in a medical tracer for gamma scintillography. For example, technicium 99, a short-lived radio-isotope, can be included in an antibiotic-metal complex as a medical tracer for gamma scintillography and used for medical imaging, for example, a site of infection in a patient.

An imaging agent can also be prepared by cross-linking the antibiotic in the chelated complex, to an anti-tumor monoclonal antibody using a hetero-bifunctional reagent, such as N-hydroxysuccinimide-activated N-propionylmaleimide. The malylated peptide antibiotic would then react with a native sulfhydryl on the antibody or a sulfhydryl introduced by treatment with a thiolating reagent such as iminothiolane. Once the peptide is grafted onto the antibody, a metal chelate of Gd or Tc, as discussed above, could be formed and used for imaging a site of a tumor in a patient. Additional heterobifunctional cross-linking agents are readily identified, for example, by referring to catalogs of reagents (e.g., the Pierce Chemical Co.).

VI. Kits

The chelated antibiotic-metal complexes of the invention can also be packaged, with appropriate written instructions, in a diagnostic kit that is useful in methods to detect organisms.

One exemplary diagnostic kit that finds utility in conducting chemiluminescent assays for microorganisms on interest, would contain the chelated complex the invention, a source of peroxide and an oxidizable substrate. For purposes of stability during storage, each kit ingredient would preferably be packaged individually and then packaged all together for sale or use.

In another embodiment, the kit could contain (a) one ore more antibiotics selected from the group consisting of glycopeptide antibiotics, ribonucleoside antibiotics, and quinolone antibiotics, and (b) a detectable transition or lanthanide metal label. Instructions could also be included for synthesizing the chelated complex, as well as for conducting the assay

### EXPERIMENTAL

The following examples are put forth so as to provide those of ordinary skill in the art with a complete disclosure and description of how to make and use the antibiotic-metal complexes disclosed and claimed herein, and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. and pressure is at or near atmospheric.

### EXAMPLE 1

### Preparation of the Cobal Complex

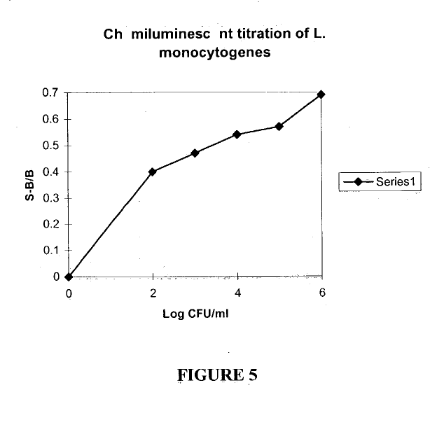

Vancomycin (purchased from Aldrich Chemical Co.), 0.05 mmoles, 0.075 g was dissolved in 8 mL of 0.05 M acetic acid. One equivalent of cobalt chloride, 12 mg, was added in 0.25 mL of the same buffer. The complex was stirred at room temperature for thirty minutes and gel filtered on Sephadex G-25 to remove unbound cobalt. The fractions absorbing at 400 nm were pooled and freeze dried. Electronic spectra of the free antibiotic and complex are depicted in FIG. 4. These results demonstrate that vancomycin readily forms a complex with cobalt, and that once formed, the complex can be easily isolated.

### EXAMPLE 2

### Chemiluminescent Cell Titration of Listeria Monocytogenes

Bacteria were diluted in sterile 0.1% peptone from cell concentrations of 10⁷ CFU/mL to 10 CFU/mL. The cells were treated with the vancomycin-Co(II) complex of Example 1 using a concentration of 10 μg/mL for twenty minutes at room temperature. Unbound complex was removed by washing, as follows: the cells were centrifuged, rinsed with 0.5 mL peptone; re-centrifuged and re-suspended in 0.1 mL peptone. Chemiluminescence was measured using 0.2 mL of Luminol reagent purchased from NEN Life Sciences (Boston, Mass.) and using a Luminator® luminometer. FIG. 5 shows the titration curve for the cells. The lowest detectable Listeria cell concentration is estimated to be 10 to 100 cells per sample.

It is to be understood that while the invention has been described in conjunction with the preferred specific embodiments thereof, the foregoing description, as well as the examples which follow, are intended to illustrate and not limit the scope of the invention. Other aspects, advantages and modifications will be apparent to those skilled in the art to which the invention pertains.

All patents, patent documents, and publications cited herein are hereby incorporated by reference in their entireties.

1. A chelated complex comprised of (a) an antibiotic selected from the group consisting of glycopeptide antibiotics, ribonucleoside antibiotics, quinolone antibiotics, and combinations thereof, and (b) a detectable label comprising a transition or lanthanide metal.

2. The complex of claim 1, wherein the label is a transition metal selected from the group consisting of Zn, Cu, Ni, Co, Fe, Mn, Cr, Tc, and their isotopes.

3. The complex of claim 2, wherein the transition metal is Co or Cr.

4. The complex of claim 1, wherein the label is a lanthanide metal selected from the group consisting of Eu, Gd, Tb, Dy, Er, Lu, and their isotopes.

5. The complex of claim 1, wherein the glycopeptide antibiotic is selected from the group consisting of actinoidin, avoparcin, balhimycins, chloroorienticins, daptomycin, ereomycin, galacardin, helevecardin, orienticins, ristocetins, ristomycin A, teicoplanin, vancomycin, and derivatives thereof.

6. The complex of claim 5, wherein the glycopeptide antibiotic is vancomycin.

7. The complex of claim 1, wherein the ribonucleoside antibiotic is a lincosamides, or a derivative thereof.

8. The complex of claim 1, wherein the quinolone antibiotic is selected from the group consisting of cinoxacin, ciprofloxacin, fleroxacin, gatifloxacin, levofloxacin, lomefloxacin, moxifloxacin, nalidixic acid, norfloxacin, ofloxacin, perfloxacin, sparfloxacin, trovafloxacin, and derivatives thereof.

9. The complex of claim 8, wherein the quinolone antibiotic is nalidixic acid.

10. The complex of claim 1, wherein the complex binds to microorganisms.

11. The complex of claim 10, wherein the microorganisms are microorganisms are selected from the group consisting of gram-positive bacteria, Mycobacteria, permeabilized gram-negative bacteria cells and protozoans.

12. A method for synthesizing a chelated antibiotic-metal complex, comprising: admixing (i) a water soluble salt of a metal selected from the group consisting of transition metals and lanthanides with (ii) an antibiotic selected from the group consisting of glycopeptide antibiotics, ribonucleoside antibiotics, and quinolone antibiotics, in (iii) a solvent for the metal salt and the antibiotic; wherein the admixing is conducted under conditions effective to promote chelation of the metal by the antibiotic, thereby forming a solution of the chelated antibiotic-metal complex.

13. The method of claim 12, wherein the glycopeptide antibiotic is selected from the group consisting of actinoidin, avoparcin, balhimycins, chloroorienticins, daptomycin, ereomycin, galacardin, helevecardin, orienticins, ristocetins, ristomycin A, teicoplanin, vancomycin, and derivatives thereof.

14. The method of claim 12, wherein the ribonucleoside antibiotic is a lincosamides, or a derivative thereof.

15. The method of claim 12, wherein the quinolone antibiotic is selected from the group consisting of cinoxacin, ciprofloxacin, fleroxacin, gatifloxacin, levofloxacin, lomefloxacin, moxifloxacin, nalidixic acid, norfloxacin, ofloxacin, perfloxacin, sparfloxacin, trovafloxacin, and derivatives thereof.

16. The method of claim 12, wherein metal is a transition metal selected from the group consisting of Zn, Cu, Ni, Co, Fe, Mn, Cr, Tc, and their isotopes.

17. The method of claim 12, wherein the metal is a lanthanide metal selected from the group consisting of Eu, Gd, Tb, Dy, Er, Lu and their isotopes.

18. The method of claim 12, wherein the solvent comprises aqueous buffer.

19. The method of claim 12, further comprising desalting the complex.

20. The method of claim 19, wherein the desalting step comprises dialysis or gel filtration.

21. The method of claim 19, further comprising isolating and drying the complex.

22. The method of claim 21, wherein the drying step comprises freeze-drying or spray drying.

23. A method for conducting a chemiluminescent assay of microorganisms in a sample comprising (a) contacting a sample with the complex of claim 1, (b) separating complex-bound microorganisms from unbound complex, (c) adding an oxidizable substrate and a source of peroxide to complex-bound microorganisms; and detecting complex-bound microorganisms by measuring luminescence.

24. The method of claim 23, wherein complex-bound microorganisms are separated using microbeads attached to a material selected from the group consisting of antibodies, bacteriophage, phage ghosts and purified phage sheath proteins.

25. The method of claim 24, wherein the microbeads are made of a material selected from the group consisting of polystyrene, latex, polymer coated ferrite, polymer coated super-paramagnetic materials, polymer coated and uncoated magnetic materials, silica, and cross-linked polysaccharides.

26. The method of claim 23, wherein the source of peroxide is hydrogen peroxide, benzoyl peroxide or cumyl peroxide.

27. The method of claim 23, wherein the source of peroxide is an enzyme selected from the group consisting of glucose oxidase and amino acid oxidases.

28. The method of claim 23, wherein the oxidizable substrate is a chemiluminescent substrate selected from the group consisting of luminol, lucigenin, penicillin, luciferin, polyaromatic phthalylhydrazides, and derivatives thereof.

29. The method of claim 23, wherein the microorganisms are selected from the group consisting of gram-positive bacteria, Mycobacteria, permeabilized gram-negative bacteria cells and protozoans.

30. The method of claim 29, wherein the microorganisms are gram-positive bacterial cells selected from the group consisting of aerobic spore-forming Bacilli, anaerobic spore-forming Bacilli, Listeria, Nocardia, Pneumococci, Staphylococci, and Streptococci.

31. The method of claim 29, wherein the microorganisms are Mycobacteria selected from the group consisting of Mycobacterium tuberculosis hominis, M. bovis, M. avium, M. paratuberculosis, and M. leprae.

32. The method of claim 29, wherein the microorganisms are permeabilized gram-negative bacterial cells selected from the group consisting of Neisseria, Flavobacter, Salmonella, and Enterobacteriaceae.

33. The method of claim 29, wherein the microorganisms are protozoans and are Plasmodia.

34. A diagnostic kit for conducting a chemiluminescent assay of microorganisms, comprising: the complex of claim 1, a source of peroxide and an oxidizable substrate.

35. The diagnostic kit of claim 34, wherein the oxidizable substrate is a chemiluminescent substrate selected from the group consisting of luminol, lucigenin, penicillin, luciferin, polyaromatic phthalylhydrazides, and derivatives thereof.

36. The diagnostic kit of claim 34, wherein the source of peroxide is hydrogen peroxide, benzoyl peroxide or cumyl peroxide.

37. The diagnostic kit of claim 34, wherein the peroxide source is an enzyme selected from the group consisting of glucose oxidase and amino acid oxidases.

38. The diagnostic kit of claim 34, wherein the transition metal in the complex is Co or Cr.

39. The diagnostic kit of claim 38, wherein the glycopeptide antibiotic in the complex is vancomycin.
